(19) United States
(12) Patent Application Publication
(10) Pub. No.: US 2020/0057056 A1
(43) Pub. Date: Feb. 20, 2020

(54) METHOD OF DETECTING OR QUANTIFYING DETECTION TARGET IN SPECIMEN, METHOD OF AGITATING REACTION MIXTURE, METHOD OF CAUSING FLOW OF LIQUID MEDIUM, ADDITIVE, REAGENT, AND AUTOMATIC ANALYZING APPARATUS

(71) Applicant: Canon Medical Systems Corporation, Otawara-shi (JP)

(72) Inventors: Satoru Sugita, Nasushiobara (JP); Hirotoshi Tahara, Nasushiobara (JP)

(73) Assignee: Canon Medical Systems Corporation, Otawara-shi (JP)

(21) Appl. No.: 16/532,768

(22) Filed: Aug. 6, 2019

(30) Foreign Application Priority Data
Aug. 7, 2018 (JP) 2018-148773
Jul. 30, 2019 (JP) 2019-139899

(51) Int. Cl. G01N 33/545; G01N 35/02; G01N 35/00; G01N 21/03; G01N 33/553

(52) U.S. Cl. CPC G01N 33/545; G01N 35/025; G01N 2035/00544; G01N 21/0303; G01N 33/553; G01N 35/0098

(57) ABSTRACT

According to one embodiment, a method of detecting or quantifying a detection target in a specimen includes: irradiating a reaction mixture with light to cause a flow of a liquid part of the reaction mixture, the reaction mixture containing: an affinity material including a carrier and an affinity substance that is carried on the carrier and has affinity to the detection target; a nanostirrer including a metal nanoparticle; and the specimen; and detecting or quantifying the detection target based on a complex composed of the affinity material and the detection target.

### CROSS-REFERENCE TO RELATED APPLICATIONS

This application is based upon and claims the benefit of priority from Japanese Patent Applications No. 2018-148773, filed Aug. 7, 2018, and No. 2019-139899, filed Jul. 30, 2019, the entire contents of all of which are incorporated herein by reference.

### FIELD

Embodiments described herein relate generally to a method of detecting or quantifying a detection target in a specimen, a method of agitating a reaction mixture, a method of causing a flow of a liquid medium, an additive, a reagent, and an automatic analyzing apparatus.

### BACKGROUND

The latex agglutination method has been performed for detecting a detection target in a specimen. The latex agglutination method is a method in which, for example, for detecting an antigen in a specimen such as a biological sample, the specimen is mixed with latex that carries an antibody or a fragment thereof specifically bound to the antigen, and the degree of latex agglutination is measured, thereby detecting or quantifying the antigen.

According to this latex agglutination method, the antigen contained in the specimen cross-links multiple latex-bound antibodies, and promotes latex agglutination. However, cross-linking does not easily occur if the amount of antigen is small, and thus latex agglutination is not sufficient to detect the agglutinates. It has been therefore difficult to quickly detect a small amount of antigen.

For shortening the time of agitating for reacting the specimen and the reagent, there has been proposed an agitation device configured to apply a sound wave within a resonance frequency band to a liquid sample containing the specimen and the reagent to cause a sound flow. However, this sound wave is generated by a sound generator attached to the wall surface of the reaction container, and requires high frequencies of several MHz to several hundred MHz to agitate the entire liquid sample in the container. Thus, there is a risk that heat denaturation occurs in a detection target such as a protein. In order to minimize damage to the specimen such as heat denaturation of a protein, there was a need to control the sound wave generation time to be instant, and this may have resulted in insufficient agitation.

### BRIEF DESCRIPTION OF THE DRAWINGS

FIG. 1A schematically shows an example of expansion/contraction motion of a metal nanoparticle;

FIG. 1B schematically shows another example of expansion/contraction motion of a metal nanoparticle;

FIG. 2 schematically shows an example of a state in a reaction mixture when a latex agglutination method is performed;

FIG. 3 is a block diagram showing an example of a functional configuration of an automatic analyzing apparatus according to the present embodiment;

FIG. 4 is a schematic diagram showing an example of a configuration of an analysis mechanism shown in FIG. 3;

FIG. 5 is a schematic diagram showing an example of an A-A cross section shown in FIG. 4; and

FIG. 6 is a graph showing reaction curves.

### DETAILED DESCRIPTION

1. Method of Detecting or Quantifying Detection Target in Specimen

According to one embodiment, a method of detecting or quantifying a detection target in a specimen includes:

irradiating a reaction mixture with light to cause a flow of a liquid part of the reaction mixture,
the reaction mixture containing:
an affinity material including a carrier and an affinity substance that is carried on the carrier and has affinity to the detection target;
a nanostirrer including a metal nanoparticle; and
the specimen; and

detecting or quantifying the detection target based on a complex composed of the affinity material and the detection target.

This method can be used for any method of detecting or quantifying a detection target in a specimen, and is particularly used for a method of detecting or quantifying a detection target in a specimen utilizing an antigen-antibody reaction (i.e., immunoassay). For example, this method can be used for an enzyme linked immunosorbent assay (ELISA) method, a chemiluminescent enzyme immunoassay (CLEIA) method, an agglutination method and the like, preferably an agglutination method, more preferably a latex agglutination method.

1-1. Reaction Mixture

The reaction mixture contains a “specimen”, an “affinity material”, and a “nanostirrer”, which will be described below in this order.

“Specimen”

The specimen is a biological sample. For example, the specimen is a body fluid or an excrement extract, specific examples of which include blood, serum, plasma, urine, lymph fluid, sputum, and a feces extract.

A detection target in the specimen is a substance used for clinical diagnosis, specific examples of which include human immunoglobulin G, human immunoglobulin M, human immunoglobulin A, human immunoglobulin E, human albumin, human fibrinogen (fibrin and its degradation product), α-fetoprotein (AFP), C-reactive protein (CRP), myoglobin, a carcinoembryonic antigen, a hepatitis virus antigen, human chorionic gonadotropin (hCG), human placental lactogen (HPL), an HIV virus antigen, an allergen, a bacterial toxin, a bacterial antigen, an enzyme, a hormone (e.g., human thyroid stimulating hormone (TSH), and insulin), a nucleic acid, a nucleic acid amplified by PCR or the like, cytokine, and a drug, contained in body fluid, urine, sputum, feces, etc.

“Affinity Material”

The affinity material includes a carrier, and an affinity substance carried on the carrier and having affinity to the detection target.

The affinity substance is preferably a substance specifically bound to the detection target. The affinity substance may be an antigen or an antibody. If the detection target is an antigen, the affinity substance may be an antibody. The antibody may be an immunoglobulin molecule of any type, and may be an immunoglobulin molecule fragment having an antigen binding site, such as Fab. While the antibody may be a monoclonal antibody or a polyclonal antibody, a monoclonal antibody that recognizes an antigenic determinant of a different antigen is preferable. Alternatively, if the detection target is an antibody, the affinity substance may be an antigen having an antigenic determinant recognized by the antibody.

The carrier that carries the affinity substance is not particularly limited, and may be of various kinds. Examples of the carrier include a carrier particle, a magnetic particle, a membrane, a well such as a microtiter well, a slide material, a plate, a microfabricated chip, a pellet, a disk, a capillary tube, a hollow fiber, a needle, and a solid fiber.

The carrier is preferably a carrier particle. For the carrier particle, those generally used in the agglutination method can be used. Examples of the carrier particle include, for example, a cellulose particle, a porous glass particle, a silica gel particle, a low and high cross-linked polystyrene particle optionally cross-linked with divinylbenzene, a grafted copolymer particle, a polyacrylamide particle, a latex particle, a dimethylacrylamide particle optionally cross-linked with N,N-bis-acryloyl ethylene diamine, and a glass particle coated with a hydrophobic polymer. Alternatively, the carrier particle may be a particle containing alkanethiolate-induced gold, polyamide, acrylic copolymer, nylon, dextran, polyacrolein, etc. The carrier particle has an average particle diameter of, for example, 20 to 800 nm, preferably 100 to 400 nm.

The carrier particle is preferably a latex particle. The latex particle refers to a carrier particle used in the latex agglutination method. For the latex particle, those publicly known may be used, an example of which may be a polystyrene-based latex particle.

The affinity material can be prepared by binding the affinity substance and the carrier. For example, if the affinity substance is an antibody or an antigen, the affinity substance can be directly bound to the carrier using an ordinary method such as a physical adsorption method or a chemical binding method. Alternatively, the affinity substance may be indirectly bound to the carrier via substances having affinity to each other (e.g., avidin and biotin, or glutathione and glutathione S-transferase).

“Nanostirrer”

The nanostirrer includes a metal nanoparticle. When the metal nanoparticle is irradiated with light while dispersed in a liquid medium, the metal nanoparticle makes motion greater than Brownian motion. When irradiated with light while dispersed in the liquid medium, the metal nanoparticle expands on the light irradiation surface by light energy, and this expansion occurs over a larger area on the light irradiation surface as compared to a non-metal because of high thermal conductivity of a metal. Thus, since the metal nanoparticle expands over a larger area only on the light irradiation surface when the metal nanoparticle is irradiated with light in the liquid medium, a momentum effectively increases by its recoil. If multiple metal nanoparticles are dispersed in the liquid medium and the momentum of each of the particles increases, a turbulent flow can be generated in the liquid medium.

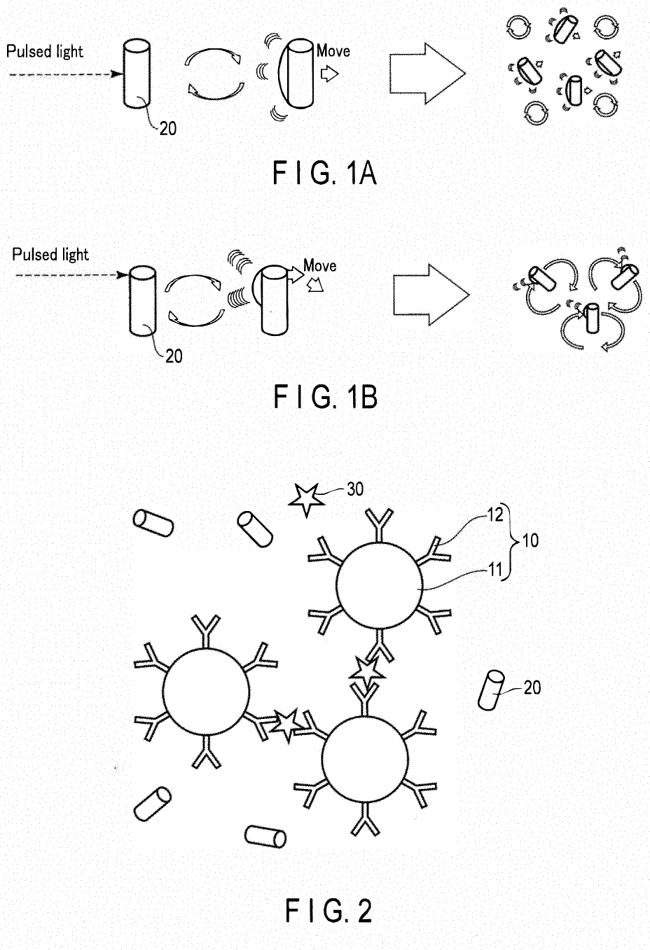

It is known that when a metal nanoparticle is irradiated with pulsed light while dispersed in a liquid medium, it expands on the light irradiation surface during irradiation, whereas it contracts (i.e., restores) back to the original form during non-irradiation. It is known that the expansion and contraction occur over a larger area on the light irradiation surface as compared to the non-metal because of high thermal conductivity of the metal. Thus, since the metal nanoparticle repeatedly expands and contracts over a larger area only on the light irradiation surface when it is irradiated with pulsed light in the liquid medium, the momentum increases effectively by its recoil. If multiple metal nanoparticles are dispersed in the liquid medium and the momentum of each of the particles increases, a turbulent flow can be generated in the liquid medium. An example of this state is shown in FIGS. 1A and 1B.

In FIGS. 1A and 1B, the metal nanoparticle has, for example, a rod shape. FIG. 1A shows the case where the side surface of the metal nanoparticle is irradiated with pulsed light. In FIG. 1A, when irradiated with pulsed light in the liquid medium, the metal nanoparticle repeatedly expands and contracts on the light irradiation surface, and this recoil moves the metal nanoparticle in a direction opposite to the light irradiation surface (right direction in FIG. 1A). If multiple metal nanoparticles are dispersed in the liquid medium and produce movement as described above, a turbulent flow is generated in the liquid medium. As a result, an agitation effect of the liquid medium can be obtained.

FIG. 1B shows the case where only the proximity of one bottom surface of the side surfaces of the metal nanoparticle is irradiated with pulsed light. Such a case may occur, for example, when pulsed light emission to the metal nanoparticle is interrupted due to the presence of another metal nanoparticle or affinity material. In FIG. 1B, in a manner similar to FIG. 1A, when irradiated with pulsed light in the liquid medium, the metal nanoparticle repeatedly expands and contracts on the light irradiation surface, and this recoil moves the metal nanoparticle in a direction opposite to the light irradiation surface (lower right direction in FIG. 1B). In FIG. 1B, the expansion and contraction occur only in the proximity of one bottom surface of the side surfaces of the metal nanoparticle; thus, a moment occurs, and rotational movement of the particle occurs. If multiple metal nanoparticles are dispersed in the liquid medium and produce rotation movement as described above, a turbulent flow is generated in the liquid medium. As a result, an agitation effect of the liquid medium can be obtained.

For the metal nanoparticle, a nanoparticle composed of any type of metal can be used. For example, a nanoparticle composed of a metal having a thermal conductivity (300K) of 20 to 450 W/mK, preferably 30 to 430 W/m·K, can be used for the metal nanoparticle. The metal nanoparticle may have any shape, and if the size is represented by an average equivalent circle diameter, the metal nanoparticle has an average equivalent circle diameter of, for example, 10 to 1000 nm, preferably 20 to 200 nm. The average equivalent circle diameter described in this specification refers to a value obtained by the following method.

A scanning electron microscope (SEM) is used to obtain an SEM image of metal nanoparticles. Next, fifty particles in which the entirety of each of the particles is visible are selected randomly from the particles shown in each SEM image, and an area of each particle selected is obtained. Each diameter of a circle having an area equivalent to the obtained areas is calculated, and then an arithmetic average of these diameters is obtained. This arithmetic average is an average equivalent circle diameter.

With such nano-level size, the metal nanoparticles can be dispersed efficiently in the reaction mixture.

The metal nanoparticle has a specific gravity of, for example, 2 to 25 g/cm³, preferably 5 to 20 g/cm³. With such specific gravity, the metal nanoparticles can efficiently agitate the liquid medium.

It is preferable that the metal nanoparticle has a shape with corners so that a turbulent flow can be efficiently generated in the liquid medium. Alternatively, it is preferable that the metal nanoparticle has a shape extending in one direction or a flat shape so that a turbulent flow can be efficiently generated in the liquid medium. That is, it is preferable that the metal nanoparticle has a rod shape or a plate shape.

The rod shape may be, for example, a column shape extending in a height direction, an elliptic column shape extending in a height direction, a quadrangular prism shape extending in a height direction, or a triangular prism shape extending in a height direction. Among them, a triangular prism shape extending in a height direction is preferable because a turbulent flow can be efficiently generated when the metal nanoparticles move. For the rod shape, a minor axis may be approx. 4 nm to approx. 9 nm, while the major axis may be approx. 10 nm to approx. 65 nm, and the ratio of the major axis length to the minor axis length may be 2 to 9.

The plate shape may be, for example, a flat column shape, a flat elliptic column shape, a flat quadrangular prism shape, or a flat triangular prism shape. Among them, a flat triangular prism shape is preferable because a turbulent flow can be efficiently generated when the metal nanoparticles move. For the plate shape, a plate thickness may be approx. 8 nm to approx. 20 nm, while a planar surface length may be approx. 20 nm to approx. 110 nm, and the ratio of the planar surface length to the plate thickness may be 2 to 11.

A material of the metal nanoparticle is not particularly limited, and is preferably a gold nanoparticle, a silver nanoparticle, or a vanadium nanoparticle. The metal nanoparticle may be one available in the market as an anisotropic noble metal nanoparticle, examples of which include a silver nanoparticle (product name: Ag-WS6-C; shape: plate shape having thickness of approx. 10 to 20 nm and planar surface length of approx. 110 nm, the plate shape including flat triangular prism shape; absorption peak wavelength: 900±30 nm; Dai Nippon Toryo Co., Ltd.), and a gold nanoparticle (product name: Au-WP7-C; shape: rod shape having minor axis length of approx. 8 nm and major axis length of approx. 65 nm; absorption peak wavelength: 1250±50 nm).

Not all of the metal nanoparticles to be used have to have the same shape or the same material, and they may be a mixture of metal nanoparticles of different shapes, or a mixture of metal nanoparticles of different materials.

The nanostirrer may include, in addition to the metal nanoparticle, an organic polymer bound to the surface of the metal nanoparticle. The organic polymer is typically of hydrophobic nature, and may be polyethylene glycol or glycidyl methacrylate. The organic polymer bound to the surface of the metal nanoparticle plays a role of moving water so as to repel a liquid part (i.e., water) of the reaction mixture. Thereby, a flow of the liquid part of the reaction mixture can be further caused. If a flow of the liquid part of the reaction mixture is further caused, this further increases the opportunity that the affinity substance and the detection target are brought into contact with each other, thereby further promoting the binding reaction and the agglutination reaction between the affinity substance and the detection target.

It is preferable that the nanostirrer is contained in the reaction mixture in an amount that allows a flow of the entire liquid part of the reaction mixture to be caused. For example, it is preferable that the nanostirrer is contained in the reaction mixture in an amount of 0.01% by mass or more with respect to the total amount of the reaction mixture excluding the nanostirrer. In order to eliminate the possibility that the presence of the nanostirrer affects the detection or quantification, it is preferable that the nanostirrer is contained in the reaction mixture in an amount of 10% by mass or less with respect to the total amount of the reaction mixture excluding the nanostirrer. That is, it is preferable that the nanostirrer is contained in the reaction mixture in an amount of 0.05 to 1% by mass with respect to the total amount of the reaction mixture excluding the nanostirrer.

“Reaction mixture” The reaction mixture contains a buffer solution as a liquid component. The total amount of the reaction mixture is not particularly limited, and may be, for example, 50 to 3000 μL, preferably 100 to 400 μL, if a small amount of detection target is detected or quantified.

The reaction mixture can be prepared by mixing, in a container, a nanostirrer-containing buffer solution, an affinity material, and a specimen. For example, the reaction mixture may be prepared by mixing the nanostirrer-containing buffer solution and the specimen, and adding the affinity material to the obtained intermediate mixture.

1-2. Light Irradiation

In this method, the reaction mixture described above is irradiated with light to cause a flow of a liquid part of the reaction mixture.

The light may be one having multiple wavelengths, but laser light is preferable. The light may be emitted continuously or intermittently, but pulsed light is preferable. Thus, pulsed laser light is more preferable.

In the case of irradation with laser light, a wavelength is preferably near the absorption peak wavelength of the metal nanoparticle, e.g., a wavelength within a range of the absorption peak wavelength±200 nm of the metal nanoparticle. The wavelength of the laser light may be determined by measuring spectral characteristics of the metal nanoparticle by an ultraviolet-visible-nearinfrared spectrophotometer. If the above-described silver nanoparticle (product name: Ag-WS6-C) is used as a metal nanoparticle, the wavelength of the laser light may be, for example, 700 to 1100 nm. If the above-described gold nanoparticle (product name: Au-WP7-C) is used as a metal nanoparticle, the wavelength of the laser light may be, for example, 1050 to 1450 nm.

In the case of irradiation with the light having multiple wavelengths, the light may include a wavelength near the absorption peak wavelength of the metal nanoparticle.

If the light is pulsed light, the pulse energy is, for example, 50 to 400 mJ, and the frequency is, for example, 0.1 to 25 MHz. The pulse waveform is, for example, a square wave. The distance from the light source to the reaction container may be, for example, 5 mm. The material of the reaction container may be, for example, glass, polycarbonate, or polystyrene.

The light does not necessarily have to be emitted to the entire reaction mixture as long as a flow of a liquid part of the reaction mixture can be caused. The beam diameter of the laser light may be, for example, 1 to 3 mm with respect to approx. 7 mm of the width of the container wall surface perpendicular to the light traveling direction. If the light is not emitted to the entire reaction mixture, only a single portion of the reaction mixture may be irradiated, or multiple portions of the reaction mixture may be irradiated.

The total light irradiation time to the reaction mixture is, for example, 2 to 60 minutes corresponding to the reaction time with the specimen.

As described above, if the metal nanoparticle is irradiated with light in the reaction mixture, a flow of a liquid part of the reaction mixture can be generated. If pulsed light is used, the metal nanoparticle repeatedly expands and contracts on the light irradiation surface, and it is therefore possible to efficiently cause a flow of a liquid part of the reaction mixture. If laser light is used, the metal nanoparticle can efficiently use the emitted light, and it is therefore possible to reduce a risk of damaging the specimen by light irradiation. Accordingly, if the pulsed laser light is used, it is possible to efficiently cause a flow of a liquid part of the reaction mixture, and to reduce the risk of damaging the specimen by light irradiation.

As will be described in the examples below, if the reaction mixture is prepared by mixing the nanostirrer-containing buffer solution and the specimen followed by adding the affinity material to the obtained intermediate mixture, the light may be emitted to the intermediate mixture in addition to emission to the reaction mixture.

1-3. Detection or Quantification

In this method, the detection target is detected or quantified based on a complex composed of the affinity material and the detection target. The complex composed of the affinity material and the detection target is, in an agglutination method, an agglutinate produced by the binding reaction and the agglutination reaction between the affinity material and the detection target.

The detection target can be detected by determining a presence or absence of the complex composed of the affinity substance and the detection target.

The presence or absence of the complex can be determined by a visual observation or a turbidity measurement. The turbidity is calculated from, for example, an absorbance based on the transmitted light intensity measured by a photometer, or a scattered light quantity based on the scattered light intensity measured by a photometer. If the turbidity is high, the complexes agglutinate, suggesting that a detection substance is present. The wavelength of the light used may be set as appropriate so as to obtain a desired detection sensitivity according to the particle diameter, etc. of the carrier particle, etc. It is preferable that the light has a wavelength within a range from a near-ultraviolet light wavelength to a near-infrared wavelength (e.g., 340 to 800 nm) in that the conventional and general device can be used.

The visual observation or turbidity measurement may be performed intermittently at a given time point or continuously over time. In addition, the determination may be made based on the difference between the turbidity measurement value at a certain time point and the turbidity measurement value at another time point.

The “turbidity measurement” in the detecting or quantifying method includes not only directly measuring the turbidity but also measuring a parameter reflecting the turbidity. The parameter may be a difference among turbidity measurement values at multiple time points, an amount of the separated agglutinate, a turbidity of non-agglutinates after separation, etc.

Quantifying the detection target can be performed by measuring the turbidity based on the complex, and calculating the amount of the detection target in the specimen based on the correlation equation between the amount of the detection target and the turbidity.

The correlation equation between the amount of the detection target and the turbidity is prepared in advance. For the measurement of the amount of the detection target and the turbidity for creating the correlation equation, if there is more data, the reliability of the correlation equation increases. The data may be those related to two or more values for the amount of the detection target, preferably three or more values for the amount of the detection target.

The correlation equation between the amount of the detection target and the turbidity may be not only the equation indicating the direct correlation between the amount of the detection target and the turbidity but also the correlation equation between the amount of the detection target and the parameter reflecting the turbidity.

The amount of the detection target in the specimen can be calculated by substituting the turbidity measurement value for the correlation equation prepared.

1-4. Latex Agglutination Method

FIG. 2 schematically shows an example of the state in the reaction mixture when the latex agglutination method is performed using the above-described method. In FIG. 2, a detection target 30 is an antigen, and an affinity material 10 is composed of a latex particle 11 and an antibody 12 carried on the latex particle 11 and specifically bound to the detection target 30. A nanostirrer 20 has a rod shape.

When the reaction mixture is irradiated with light, the nanostirrer 20 causes a flow of a liquid part of the reaction mixture. If the detection target 30 is present in the specimen, the flow of the liquid part of the reaction mixture promotes the binding reaction between the detection target 30 and the antibody 12 carried on the latex particle 11, and also promotes the agglutination reaction of the latex particle 11.

1-5. Advantageous Effects

According to the method described above, the metal nanoparticle is used to cause a flow of the liquid part of the reaction mixture, and this can increase the opportunity that the affinity substance and the detection target are brought into contact with each other, and promote the binding reaction between the affinity substance and the detection target. Therefore, it is possible to quickly detect or quantify the detection target in the specimen. In particular, according to the method described above, the reaction mixture is irradiated with light to cause a flow of a liquid part of the reaction mixture, and therefore a flow of the liquid can be quickly caused. Furthermore, because the flow of the liquid caused by the nanostirrer is gentle, the complex once formed by the binding reaction is not decomposed by reverse reactions of these reactions.

Thus, according to the above-described method, the detection accuracy can be increased even if the amount of the detection target is very small. Moreover, the time required for binding, agglutination, etc. can be shortened. Furthermore, even if the reaction field is extremely narrow, the binding between the affinity substance and the detection target can be promoted, and therefore, the above-described method can be used not only for reaction in cells, wells, etc. as conventionally performed, but also for reaction in a microchemical process, a microchannel, a microreactor, etc.

In addition, according to the above-described method, the momentum of the nanostirrer is increased by low-energy light irradiation, and this can cause a flow of a liquid part of the reaction mixture. On the other hand, if the agglutination reaction is promoted by moving the affinity material including the carrier particle and the affinity substance by light irradiation, this causes a cavitation (i.e., a physical phenomenon in which pressure difference in a liquid flow leads to formation and collapse of bubbles in a short period of time) at an ambient environment of the affinity material in the reaction mixture. A shock wave is generated when bubbles collapse, which may cause protein denaturation or decomposition of the complex once formed by the binding reaction and the agglutination reaction. Since the above-described method can be performed by increasing the momentum of the nanostirrer by low-energy light irradiation, no cavitation occurs at an ambient environment of the affinity material, and it is therefore possible to suppress damage to the specimen or decomposition of the reaction product.

In addition, the above-described method can be performed by adding a light irradiation unit to an existing analyzing apparatus, and thus the above-described effect can be achieved with simple device structures.

2. Additive and Reagent

According to another aspect, there is provided an additive for agitating a reaction mixture for detecting or quantifying a detection target in a specimen, in which the additive includes a nanostirrer that includes a metal nanoparticle.

The additive includes the nanostirrer described in the “Nanostirrer” section. The additive can be used for agitating a reaction mixture by addition to the reaction mixture for detecting or quantifying a detection target in a specimen.

According to another aspect, there is provided a reagent for preparing a reaction mixture for detecting or quantifying a detection target in a specimen, in which the reagent includes a buffer solution and a nanostirrer that includes a metal nanoparticle.

The reagent includes the buffer solution constituting the reaction mixture, and the nanostirrer described in the “Nanostirrer” section. The reagent can be used for preparing a reaction mixture for detecting or quantifying a detection target in a specimen. The reagent can be used in combination with another reagent including the “affinity material” described above. Therefore, the reagent including the buffer solution and the nanostirrer may form a kit in combination with another reagent including the “affinity material” described above. In the following description, the reagent including the buffer solution and the nanostirrer will also be referred to as a “first reagent”, while the reagent including the affinity material will also be referred to as a “second reagent”.

3. Method of Agitating Reaction Mixture and Method of Causing Flow of Liquid Medium

When only the light irradiation step of “1. Method of detecting or quantifying detection target in specimen” described above is performed, it is possible to agitate a reaction mixture for detecting or quantifying a detection target in a specimen. Thus, according to another aspect, there is provided a method of agitating a reaction mixture for detecting or quantifying a detection target in a specimen, in which the method includes the light irradiation step of “1. Method of detecting or quantifying detection target in specimen” described above. That is, according to another aspect, there is provided a method of agitating a reaction mixture for detecting or quantifying a detection target in a specimen, in which the method includes:

irradiating a reaction mixture with light to cause a flow of a liquid part of the reaction mixture, in which the reaction mixture contains:
an affinity material including a carrier and an affinity substance that is carried on the carrier and has affinity to the detection target;
a nanostirrer including a metal nanoparticle; and
the specimen.

Thus, the technique of “1. Method of detecting or quantifying detection target in specimen” described above is applicable to any method of agitating a reaction mixture.

Moreover, with the technique of “1. Method of detecting or quantifying detection target in specimen” described above, it is possible to cause a flow of the liquid medium by dispersing the nanostirrers in the liquid medium and irradiating this dispersion liquid with light. That is, according to another aspect, there is provided a method of causing a flow of a liquid medium, in which the method includes:

irradiating a dispersion liquid with light to cause a flow of the liquid medium, in which the dispersion liquid contains:
the liquid medium; and
a nanostirrer dispersed in the liquid medium and including a metal nanoparticle.

Thus, the technique of “1. Method of detecting or quantifying detection target in specimen” described above is applicable to any method of causing a flow of a liquid medium.

4. Automatic Analyzing Apparatus

According to another aspect, there is provided an automatic analyzing apparatus that subjects a reaction mixture containing a specimen, an affinity material, and a nanostirrer to, for example, optical measurement to analyze a detection target in the specimen, in which the automatic analyzing apparatus includes a light irradiating unit that emits light to cause a flow of a liquid part of the reaction mixture. A “detection target in a specimen” can be selected as appropriate in accordance with a test item. The word “analyzing/analysis” is used as a general term for detection and quantification throughout this specification.

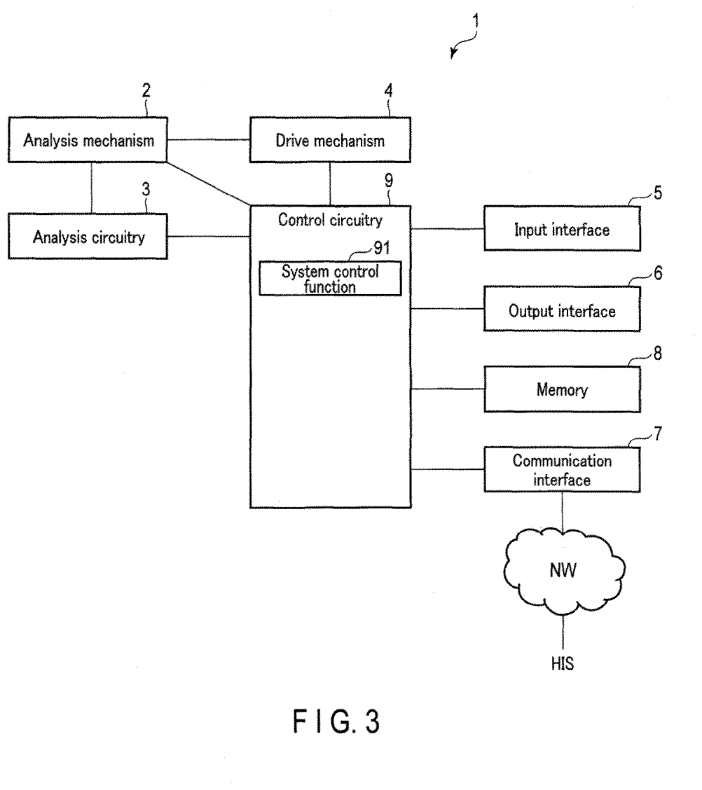

FIG. 3 is a block diagram showing an example of a functional configuration of an automatic analyzing apparatus 1 according to the present embodiment. In FIG. 3, the automatic analyzing apparatus 1 includes an analysis mechanism 2, analysis circuitry 3, a drive mechanism 4, an input interface 5, an output interface 6, a communication interface 7, a memory 8, and control circuitry 9.

The analysis mechanism 2 mixes a specimen, a first reagent, and a second reagent. The first reagent contains a buffer solution constituting the reaction mixture, and the nanostirrer described in the “Nanostirrer” section. The second reagent includes the “affinity material” described above. The analysis mechanism 2 measures the reaction mixture composed of the specimen, the first reagent, and the second reagent, and generates subject data which may be represented as, for example, an absorbance. The analysis mechanism 2 further measures the reaction mixture composed of the standard sample, the first reagent, and the second reagent, and generates standard data which may be represented as, for example, an absorbance.

The analysis circuitry 3 is a processor to analyze the standard data and the subject data generated by the analysis mechanism 2, and to generate calibration data, analysis data, etc. The analysis circuitry 3 reads operation programs from the memory 8, and generates the calibration data, the analysis data, etc., in accordance with the read operation programs. The analysis circuitry 3 generates, based on the standard data, the calibration data indicating the relation between the standard data and the standard value that is preset for the standard sample. The analysis circuitry 3 generates the analysis data which may be represented as, for example, a concentration value and an enzyme activity value, based on subject data and the calibration data with a test item corresponding to this subject data. The analysis circuitry 3 outputs the generated calibration data, analysis data, etc., to the control circuitry 9.

The drive mechanism 4 drives the analysis mechanism 2 under the control of the control circuitry 9. The drive mechanism 4 is configured with, for example, a gear, a stepping motor, a belt conveyor, a lead screw, and so on.

The input interface 5 accepts, for example, settings of assay parameters, etc., for each test item intended for a measurement-requested sample, from an operator or via an in-hospital network NW. The input interface 5 is connected to an input device such as a mouse, a keyboard, a touch pad which receives input instructions through contact on its operation screen, touch screen in which a display screen and a touch pad are integrated, non-contact input circuitry using an optical sensor, sound input circuitry, etc. The input interface 5 is connected to the control circuitry 9 so that it converts operational instructions, input by an operator, into electric signals and outputs them to the control circuitry 9. In the present disclosure, the input interface 5 is not limited to physical operation components such as a mouse and a keyboard. Examples of the input interface 5 may also include circuitry for electric signals, which is adapted to receive an electric signal corresponding to the operational instruction input from an external input device separate from the automatic analyzing apparatus 1, and to output the electric signal to the control circuitry 9.

The output interface 6 is connected to the control circuitry 9 and outputs the signals coming from the control circuitry 9. The output interface 6 is connected to an output device such as a display, print circuitry, and a sound device. The display may be a liquid crystal display, an organic EL display, an LED display, a plasma display, a CRT display, etc. Also, the output interface 6 may include circuitry for converting data of a display object into video signals and supplying the video signals to external entities. The print circuitry may be a printer, etc. The output interface 6 may also include circuitry for supplying data of a print object to external entities. The sound device may be a speaker, etc. The output interface 6 may also include circuitry for supplying sound signals to external entities.

The communication interface 7 is connected to, for example, the in-hospital network NW. The communication interface 7 performs data communication with a hospital information system (HIS) via the in-hospital network NW. It is also possible for the communication interface 7 to perform data communication with the HIS via a laboratory information system (LIS) connected to the in-hospital network NW.

The memory 8 may be, for example, a processor-readable storage medium such as a magnetic or optical storage medium or a semiconductor memory. It is not necessary to realize the memory 8 by a single storage medium. For example, the memory 8 may be configured with multiple storage media.

The memory 8 stores operation programs for the analysis circuitry 3 to be executed, as well as operation programs for the control circuitry 9 to be executed. The memory 8 stores, for each test item, the calibration data generated by the analysis circuitry 3. The memory 8 stores, for each subject sample, the analysis data generated by the analysis circuitry 3. The memory 8 stores a test order input by an operator, or a test order received by the communication interface 7 via the in-hospital network NW.

The control circuitry 9 is a processor functioning as a center of the automatic analyzing apparatus 1. The control circuitry 9 executes the operation programs stored in the memory 8 to realize functions corresponding to the operation programs. The control circuitry 9 may be provided with a storage area for storing at least part of the data stored in the memory 8.

The control circuitry 9 runs the operation programs stored in the memory 8 to provide a system control function 91, for example. Note that the present embodiment will be described assuming that a single processor realizes the system control function 91, but the embodiment is not limited to such a configuration. For example, multiple independent processors may be used in combination to form control circuitry to have the respective processors run the operation programs, so that the system control function 91 will be performed.

The system control function 91 is a function to take total control over the components of the automatic analyzing apparatus 1, based on the input information via the input interface 5.

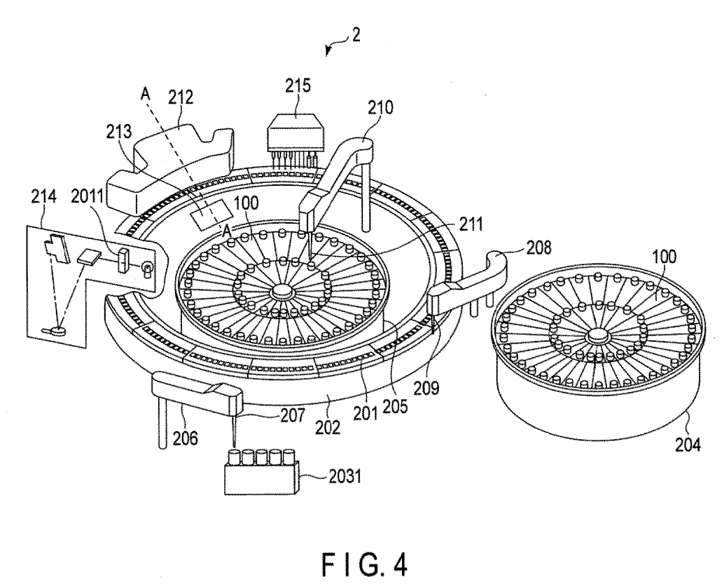

FIG. 4 is a schematic diagram showing an example of a configuration of an analysis mechanism 2 shown in FIG. 3. In FIG. 4, the analysis mechanism 2 includes a reaction disk 201, a thermostat bath 202, a first-reagent carousel 204, and a second-reagent carousel 205.

The reaction disk 201 conveys reaction containers 2011 along a predetermined path. More specifically, the reaction disk 201 holds the multiple reaction containers 2011 in an annular arrangement. The reaction disk 201 is rotated and stopped alternately at regular time intervals by the drive mechanism 4.

The thermostat bath 202 stores a thermal medium that is set at a predetermined temperature. By immersing the reaction containers 2011 in the stored thermal medium, the thermostat bath 202 increases the temperature of the reaction mixture contained in the reaction containers 2011.

The first-reagent carousel 204 is adapted for cold storage of multiple reagent containers 100 that contain a first reagent. The first-reagent carousel 204 is covered with a detachable reagent cover (not shown in FIG. 4) for cold storage, evaporation prevention, etc. The first-reagent carousel 204 encloses reagent racks in such a manner that they are able to turn. The reagent racks hold the multiple reagent containers 100 in an annular arrangement. The reagent racks are rotated by the drive mechanism 4.

A predetermined first-reagent aspiration position is provided above the first-reagent carousel 204. The first-reagent aspiration position is set at, for example, the intersection between the rotational trajectory of a first-reagent dispensing probe 209 and the traveling path of the openings of the reagent containers 100 annularly arranged by the reagent racks.

The second-reagent carousel 205 is adapted for cold storage of multiple reagent containers 100 that contain a second reagent. The second-reagent carousel 205 is covered with a detachable reagent cover (not shown in FIG. 4). The second-reagent depository 205 encloses reagent racks in such a manner that they are able to turn. The reagent racks hold the multiple reagent containers 100 in an annular arrangement.

A predetermined second-reagent aspiration position is provided above the second-reagent carousel 205. The second-reagent aspiration position is set at, for example, the intersection between the rotational trajectory of a second-reagent dispensing probe 211 and the traveling path of the openings of the reagent containers 100 annularly arranged by the reagent racks.

In FIG. 4, the analysis mechanism 2 further includes a sample dispensing arm 206, a sample dispensing probe 207, a first-reagent dispensing arm 208, the first-reagent dispensing probe 209, a second-reagent dispensing arm 210, the second-reagent dispensing probe 211, a stirring unit 212, a light irradiation unit 213, a photometry unit 214, and a washing unit 215.

The sample dispensing arm 206 is provided on the outer periphery of the reaction disk 201. The sample dispensing arm 206 is adapted so that it can vertically ascend and descend, and also horizontally rotate, by the drive mechanism 4. The sample dispensing arm 206 holds the sample dispensing probe 207 at its one end.

The sample dispensing probe 207 rotates along the arc rotational trajectory in conjunction with the rotation of the sample dispensing arm 206. This rotational trajectory includes a sample aspiration position that is provided for aspirating a part (i.e., a specimen) of the sample contained in the sample containers held by a sample rack 2031. The sample rack 2031 can hold multiple sample containers containing measurement-requested samples, and is conveyed in the proximity of the sample dispensing probe 207. The sample aspiration position is set at, for example, the intersection between the rotational trajectory of the sample dispensing probe 207 and the traveling path of the openings of the sample containers held by the sample rack 2031. The rotational trajectory of the sample dispensing probe 207 includes a sample discharge position that is provided for discharging the specimen aspirated by the sample dispensing probe 207 to the reaction containers 2011. The sample discharge position corresponds to the intersection between the rotational trajectory of the sample dispensing probe 207 and the traveling path of the reaction containers 2011 held by the reaction disk 201.

The sample dispensing probe 207 is driven by the drive mechanism 4 so that it descends into the sample container at the sample aspiration position, and aspirates the specimen from the sample contained in the sample container under the control of the control circuitry 9. The sample dispensing probe 207 is driven by the drive mechanism 4 so that it ascends to the top dead point at the sample aspiration position. The sample dispensing probe 207 is driven by the drive mechanism 4 so that it descends into the reaction container 2011 directly below the sample discharge position, and discharges the aspirated specimen to the reaction container 2011 under the control of the control circuitry 9. The sample dispensing probe 207 is driven by the drive mechanism 4 so that it ascends to the top dead point at the sample discharge position.

The first-reagent dispensing arm 208 is provided between the reaction disk 201 and the first-reagent carousel 204. The first-reagent dispensing arm 208 is adapted so that it can vertically ascend and descend, and also horizontally rotate, by the drive mechanism 4. The first-reagent dispensing arm 208 holds the first-reagent dispensing probe 209 at its one end.

The first-reagent dispensing probe 209 rotates along the arc rotational trajectory in conjunction with the rotation of the first-reagent dispensing arm 208. This rotational trajectory includes the first-reagent aspiration position. The rotational trajectory of the first-reagent dispensing probe 209 includes a first-reagent discharge position that is set for discharging the reagent aspirated by the first-reagent dispensing probe 209 to the reaction containers 2011. The first-reagent discharge position corresponds to the intersection between the rotational trajectory of the first-reagent dispensing probe 209 and the traveling path of the reaction containers 2011 held by the reaction disk 201.

The first-reagent dispensing probe 209 is driven by the drive mechanism 4 so that it descends into the reagent container 100 directly below the first-reagent aspiration position, and aspirates the first reagent in the reagent container 100 under the control of the control circuitry 9. The first-reagent dispensing probe 209 is driven by the drive mechanism 4 so that it ascends to the top dead point at the first-reagent aspiration position. The first-reagent dispensing probe 209 is driven by the drive mechanism 4 so that it descends into the reaction container 2011 directly below the first-reagent discharge position, and discharges the aspirated first reagent to the reaction container 2011 under the control of the control circuitry 9. The first-reagent dispensing probe 209 is driven by the drive mechanism 4 so that it ascends to the top dead point at the first-reagent discharge position.

The second-reagent dispensing arm 210 is provided between the reaction disk 201 and the second-reagent carousel 205. The second-reagent dispensing arm 210 is adapted so that it can vertically ascend and descend, and also horizontally rotate, by the drive mechanism 4. The second-reagent dispensing arm 210 holds the second-reagent dispensing probe 211 at its one end.

The second-reagent dispensing probe 211 rotates along the arc rotational trajectory in conjunction with the rotation of the second-reagent dispensing arm 210. This rotational trajectory includes the second-reagent aspiration position. The rotational trajectory of the second-reagent dispensing probe 211 includes a second-reagent discharge position that is set for discharging the reagent aspirated by the second-reagent dispensing probe 211 to the reaction containers 2011. The second-reagent discharge position corresponds to the intersection between the rotational trajectory of the second-reagent dispensing probe 211 and the traveling path of the reaction containers 2011 held by the reaction disk 201.

The second-reagent dispensing probe 211 is driven by the drive mechanism 4 so that it descends into the reagent container 100 directly below the second-reagent aspiration position, and aspirates the second reagent in the reagent container 100 under the control of the control circuitry 9. The second-reagent dispensing probe 211 is driven by the drive mechanism 4 so that it ascends to the top dead point at the second-reagent aspiration position. The second-reagent dispensing probe 211 is driven by the drive mechanism 4 so that it descends into the reaction container 2011 directly below the second-reagent discharge position, and discharges the aspirated second reagent to the reaction container 2011 under the control of the control circuitry 9. The second-reagent dispensing probe 211 is driven by the drive mechanism 4 so that it ascends to the top dead point at the second-reagent discharge position.

The stirring unit 212 is provided near the outer periphery of the reaction disk 201. The stirring unit 212 includes a stirrer that stirs the specimen and the first reagent contained in the reaction container 2011, or the specimen, the first reagent, and the second reagent contained in the reaction container 2011, at a stirring position on the reaction disk 201.

The light irradiating unit 213 is provided at a position facing the stirring unit 212 on the inner periphery side of the reaction disk 201. The light irradiating unit 213 includes, for example, a laser oscillator 2131. The laser oscillator 2131 is an example of a light source that generates laser light (preferably pulsed laser light) having a predetermined wavelength. The wavelength of the laser light is preferably a wavelength near the absorption peak wavelength of the metal nanoparticle as described above.

The light source of the light irradiating unit 213 is not limited to the laser oscillator 2131. The light irradiating unit 213 may include a light source that generates light having multiple wavelengths. In this case, the light generated from the light source may include a wavelength near the absorption peak wavelength of the metal nanoparticle.

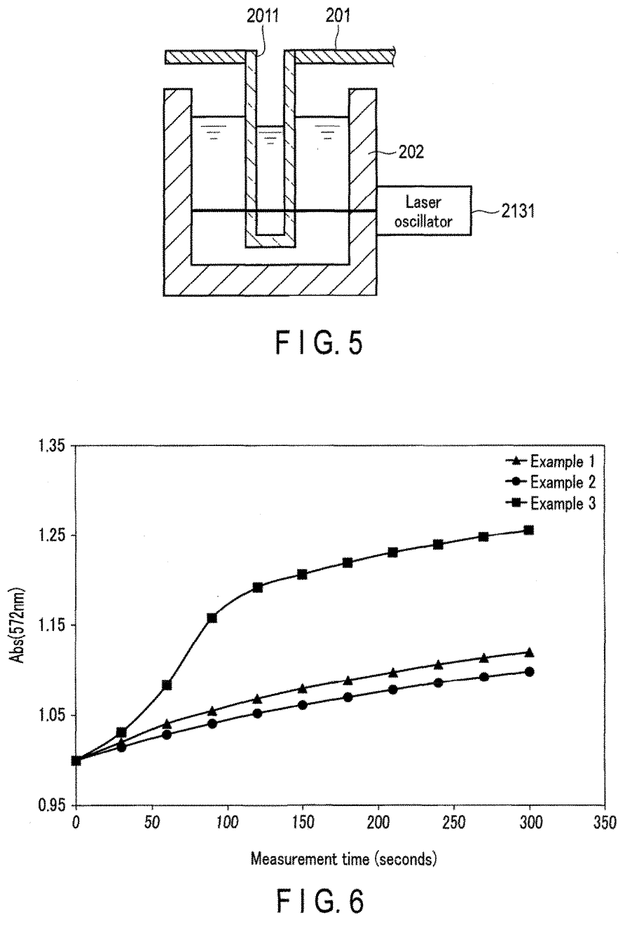

FIG. 5 is a schematic diagram showing an example of an A-A cross section shown in FIG. 4. The laser light generated by the laser oscillator 2131 enters the thermostat bath 202 from a window provided on the wall surface on the inner peripheral side of the thermostat bath 202. The laser light that has entered the thermostat bath 202 is emitted to a predetermined area on the wall surface of the reaction container 2011. The metal nanoparticles in the reaction mixture within the area irradiated with the laser light expand on the light irradiation surfaces of the particles. In the case of pulsed laser light, the metal nanoparticles repeatedly expand and contract on the light irradiation surfaces of the particles. Thereby, a flow of the liquid part of the reaction mixture is efficiently caused.

FIG. 5 has been described assuming the case where only a single portion of the reaction container 2011 is irradiated with the laser light. However, the configuration is not limited to this. The laser oscillator 2131 may generate multiple laser light beams to irradiate the reaction container 2011 with the multiple laser light beams.

The stirring unit 212 and the light irradiating unit 213 are provided to face each other with the reaction disk 201 therebetween, and this allows simultaneously performing stirring by the stirrer of the stirring unit 212 and stirring by the laser light generated by the light irradiating unit 213. Thereby, the stirring effect can be enhanced. In addition, it is possible to switch stirring by the stirrer of the stirring unit 212 and stirring by the laser light generated by the light irradiating unit 213. As a result, it is possible to perform stirring suitable for reaction types.

FIG. 4 has been described assuming the case where only one light irradiating unit 213 is provided. However, the configuration is not limited to this. A plurality of light irradiating units 213 may be provided on the circumference. Thereby, the time of emitting the laser light to one reaction container can be longer. That is, the stirring time can be longer.

The photometry unit 214 optically measures a reaction product in the reaction mixture of the specimen and the reagent discharged into the reaction containers 2011. The photometry unit 214 includes a light source and a photodetector. The photometry unit 214 emits light from the light source under the control of the control circuitry 9. The emitted light enters the reaction container 2011 through the first sidewall, and exits the reaction container 2011 through the second sidewall facing the first sidewall. The photometry unit 214 detects the light coming out of the reaction container 2011 by the photodetector.

More specifically, and for example, the photodetector detects the light having passed through the reaction mixture of the standard sample, the first reagent, and the second reagent in the reaction container 2011, and generates standard data represented as an absorbance, etc. based on the intensity of the detected light. The photodetector also detects the light having passed through the reaction mixture of the specimen, the first reagent, and the second reagent in the reaction container 2011, and generates subject data represented as an absorbance, etc. based on the intensity of the detected light. The photometry unit 214 outputs the generated standard data and subject data to the analysis circuitry 3.

The washing unit 215 washes the inside of the reaction containers 2011 for which the measurement of the reaction mixture by the photometry unit 214 has been completed.

The above-described automatic analyzing apparatus irradiates the reaction mixture containing the nanostirrer with light having a predetermined wavelength. As described above, the nanostirrer irradiated with the light causes a flow of a liquid part of the reaction mixture. If there is a detection target in the specimen, the flow of the liquid part of the reaction mixture promotes the binding reaction between the detection target and the affinity substance carried on the carrier particle, and also promotes the agglutination reaction of the carrier particle.

### Examples

The method of quantifying C-reactive protein (CRP) in serum or plasma was performed in the following manner.

The in-vitro diagnostic, CRP Auto “TBA”, available as the C-reactive protein kit in the market was used.

Measurement Target

CRP standard solution “TBA” for latex (CRP concentration: 8 mg/dL)

Nanostirrer

Anisotropic noble metal nanoparticle Ag-WS6-C (Dai Nippon Toryo Co., Ltd.) (plate shape, thickness: 10-20 nm, planar surface length: 110 nm)

### 1. Device

Discrete clinical chemistry automatic analyzer TBA-120FR (Canon Medical Systems Corporation)

### 2. Assay Parameter

Sample (CRP standard solution described above); 3.0 μL

Reagent 1 (buffer solution); 150 μL

Reagent 2 (anti-human CRP polyclonal antibody (rabbit)-bound latex suspension); 150 μL

Photometric wavelength (572 nm) response curve acquired

### 3. Test Protocol

### Example 1 (Piezo Agitation)

The Sample (CRP standard solution described above) was dispensed by the sample dispensing probe into the reaction container (glass tube). Reagent 1 (hereinafter, R1) was dispensed by the first-reagent dispensing probe into the reaction container into which the Sample was dispensed, and stirred with the piezo stirrer provided in the stirring unit. After elapse of a predetermined time from the agitation by the stirring unit, Reagent 2 (hereinafter, R2) was dispensed by the second-reagent dispensing probe into the reaction container containing the mixture of the Sample and R1, and stirred with the stirrer unit.

After the agitation by the stirrer unit, the reaction container containing the reaction mixture of the Sample, R1, and R2 was irradiated with the light from the light source provided in the photometry unit, and the light that had passed through the reaction container was detected by the photodetector. The detection was conducted every 30 seconds for 5 minutes. The absorbance was calculated based on the intensity of the transmitted light detected, and the ratio to the absorbance at the detection start time was calculated. The results are shown in FIG. 6.

### Example 2 (Addition of Nanostirrer)

The nanostirrer (Ag-WS6-C) was added so as to be 0.1% by mass in R1, thereby preparing R1 containing the nanostirrer (hereinafter, R1-AgWS6).

The Sample (CRP standard solution described above) was dispensed by the sample dispensing probe into the reaction container (glass tube). R1-AgWS6 was dispensed by the first-reagent dispensing probe into the reaction container into which the Sample was dispensed, and the resulting intermediate mixture was not stirred with the stirring unit. After elapse of a predetermined time from the dispensing of R1-AgWS6, R2 was dispensed by the second-reagent dispensing probe into the reaction container containing the intermediate mixture, and the resulting reaction mixture was not stirred with the stirring unit.

The reaction container containing the reaction mixture of the Sample, R1-AgWS6 and R2 was irradiated with the light from the light source provided in the photometry unit, and the light that had passed through the reaction container was detected by the photodetector. The detection was conducted every 30 seconds for 5 minutes after R2 was dispensed. The absorbance was calculated based on the intensity of the transmitted light detected, and the ratio to the absorbance at the detection start time was calculated. The results are shown in FIG. 6.

### Example 3 (Addition of Nanostirrer and Light Irradiation)

The nanostirrer (Ag-WS6-C) was added so as to be 0.1% by mass in R1, thereby preparing R1 containing the nanostirrer (hereinafter, R1-AgWS6).

The Sample (CRP standard solution described above) was dispensed by the sample dispensing probe into the reaction container (glass tube). R1-AgWS6 was dispensed by the first-reagent dispensing probe into the reaction container into which the Sample was dispensed, and the resulting intermediate mixture was not stirred with the stirring unit. After elapse of a predetermined time from the dispensing of R1-AgWS6, R2 was dispensed by the second-reagent dispensing probe into the reaction container containing the intermediate mixture, and the resulting reaction mixture was not stirred with the stirring unit. Instead, the area below the photometric point of the reaction container was irradiated for 5 minutes with the pulsed laser light (YAG laser, pulse energy: 50 to 400 mJ, wavelength: 1064 nm, beam diameter: 2 mm, distance from light source to reaction container: 5 mm).

The reaction container containing the reaction mixture of the Sample, R1-AgWS6 and R2 was irradiated with the light from the light source provided in the photometry unit, and the light that had passed through the reaction container was detected by the photodetector. The detection was conducted every 30 seconds over the irradiation period of the pulsed laser light. However, the irradiation with the pulsed laser light was stopped at the time of detection. The absorbance was calculated based on the intensity of the transmitted light detected, and the ratio to the absorbance at the detection start time was calculated. The results are shown in FIG. 6.

### 4. Results

As shown in FIG. 6, a clear reaction promotion effect was exhibited when the nanostirrer was added and the irradiation with pulsed laser light was performed, as compared to when the piezo agitation was performed or when only the nanostirrer was added. There was no significant difference in the reaction curve when only the nanostirrer was added as compared when the piezo agitation was performed. These results show that a reaction promotion effect can be obtained when light irradiation is performed after the addition of the nanostirrer.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A method of detecting or quantifying a detection target in a specimen, the method comprising:
irradiating a reaction mixture with light to cause a flow of a liquid part of the reaction mixture,
the reaction mixture containing:
an affinity material including a carrier and an affinity substance that is carried on the carrier and has affinity to the detection target;
a nanostirrer including a metal nanoparticle; and
the specimen; and
detecting or quantifying the detection target based on a complex composed of the affinity material and the detection target.

2. The method according to claim 1, wherein the carrier is a carrier particle.

3. The method according to claim 2, wherein the carrier particle is a latex particle.

4. The method according to claim 1, wherein the light is pulsed light.

5. The method according to claim 1, wherein the light is pulsed laser light.

6. The method according to claim 1, wherein the metal nanoparticle has a rod shape or a plate shape.

7. The method according to claim 1, wherein the metal nanoparticle is a gold nanoparticle or a silver nanoparticle.

8. The method according to claim 1, wherein the affinity substance is an antigen or an antibody.

9. The method according to claim 1, wherein the nanostirrer includes the metal nanoparticle and an organic polymer that is bound to a surface of the metal nanoparticle.

10. An additive for agitating a reaction mixture for detecting or quantifying a detection target in a specimen, the additive comprising a nanostirrer that includes a metal nanoparticle.

11. A reagent for preparing a reaction mixture for detecting or quantifying a detection target in a specimen, the reagent comprising a buffer solution and a nanostirrer that includes a metal nanoparticle.

12. A method of agitating a reaction mixture for detecting or quantifying a detection target in a specimen, the method comprising:
irradiating a reaction mixture with light to cause a flow of a liquid part of the reaction mixture,
the reaction mixture containing:
an affinity material including a carrier and an affinity substance that is carried on the carrier and has affinity to the detection target;
a nanostirrer including a metal nanoparticle; and
the specimen.

13. A method of causing a flow of a liquid medium, the method comprising:
irradiating a dispersion liquid with light to cause a flow of the liquid medium,
the dispersion liquid containing:
the liquid medium; and
a nanostirrer dispersed in the liquid medium and including a metal nanoparticle.

14. An automatic analyzing apparatus comprising:
a first light source that irradiates a reagent container containing a reaction mixture with first light to cause a flow of a liquid part of the reaction mixture,
the reaction mixture containing:
an affinity material including a carrier and an affinity substance that is carried on the carrier and has affinity to a detection target in a specimen;
a nanostirrer including a metal nanoparticle; and
the specimen;
a second light source that irradiates the reagent container with second light; and
a photodetector that detects light exiting the reagent container irradiated with the second light.
